(19) United States
(12) Patent Application Publication
(10) Pub. No.: US 2022/0287329 A1
(43) Pub. Date: Sep. 15, 2022

(54) FEED ADDITIVE FORMULATION FOR FISH AND METHODS OF MAKING AND USING THE SAME

(71) Applicant: BioResource International, Inc., Durham, NC (US)

(72) Inventors: Jeng-Jie WANG, Durham, NC (US); Giles SHIH, Durham, NC (US)

(21) Appl. No.: 17/637,500

(22) PCT Filed: Aug. 25, 2020
(86) PCT No.: PCT/US20/47710

§ 371 Date: Feb. 23, 2022

Related U.S. Application Data
(60) Provisional application No. 62/893,832, filed on Aug. 30, 2019.

(51) Int. Cl. A23K 10/18; A23K 50/80; A23K 20/163

(52) U.S. Cl. CPC A23K 10/18; A23K 50/80; A23K 20/163

(57) ABSTRACT

The presently disclosed subject matter relates to feed additive formulations for fish. The disclosed formulations may comprise an isolated xylanase enzyme and a B. licheniformis strain PWD-1. The feed additive formulations may further include B. amyloliquefaciens strain Ba-BPD1. The disclosed formulations are useful for addition to feeds for fish to synergistically improve the performance of the fish.

### CROSS REFERENCE TO RELATED APPLICATIONS

This application claims priority to U.S. Provisional Patent Application No. 62/893,832, filed Aug. 30, 2019, the entire contents of which is incorporated by reference herein.

### FIELD OF INVENTION

The presently disclosed subject matter relates generally to a feed additive formulation for fish and to methods of making and using the disclosed formulation.

### BACKGROUND

For decades, animal production has depended on antibiotic growth promotors (AGPs) to maintain animal health and improve productivity. With the removal of AGPs from animal production in many countries, producers have attempted to use various feed additives, such as organic acids, enzymes, and probiotics with varying levels of efficacy and inconsistent results. Particularly, many enzyme and probiotic products currently used in the market are costly and have been proven to be unstable when used in industry feed and animal production processes. Moreover, livestock producers seek to reduce feed costs by utilizing alternative feed ingredients or by replacing expensive nutrients via supplementation of various feed additives. One reformulation strategy that is gaining attention in aqua production is the reduction of dietary energy, coupled with supplementation of carbohydrases and/or direct-fed microbials (DFMs), additives that may improve the nutritive value, digestibility and utilization of feed. The combinational use of xylanase and DFMs has been shown to improve gut function and nutrient digestibility in poultry and swine, allowing for improved production performance and feed costs savings. However, the combination of xylanase and DFMs as ingredients for fish feed or fish feed additives has not been used. It would be beneficial to provide combinations of enzymes and probiotics that improve production performance characteristics of fish when added to fish feeds.

### SUMMARY

In some embodiments, the presently disclosed subject matter is directed to a feed additive formulation for fish feeds. Particularly, the formulation comprises an isolated xylanase enzyme and a biologically pure culture of Bacillus licheniformis strain PWD-1 (Accession No. 53757) or a mutant having all of the identifying characteristics thereof. In some embodiments, the formulation further comprises a biologically pure culture of Bacillus amyloliquefaciens strain Ba-BPD1 (Accession No. DSM 21836) or a mutant having all the identifying characteristics thereof.

In some embodiments, the presently disclosed subject matter is directed to a feed composition for fish comprising an isolated xylanase enzyme and a biologically pure culture of Bacillus licheniformis strain PWD-1 (Accession No. 53757) or a mutant having all of the identifying characteristics thereof. In some embodiments, the feed composition further comprises a biologically pure culture of Bacillus amyloliquefaciens strain Ba-BPD1 (Accession No. DSM 21836) or a mutant having all the identifying characteristics thereof.

In some embodiments, the presently disclosed subject matter is directed to a method of increasing the performance of a fish. The method comprises administering to the fish an effective amount of a feed composition comprising a xylanase enzyme and a biologically pure culture of Bacillus licheniformis strain PWD-1 (Accession No. 53757) or a mutant having all the identifying characteristics thereof. In some embodiments of the method, the feed composition further comprises a biologically pure culture of Bacillus amyloliquefaciens strain Ba-BPD1 (Accession No. DSM 21836) or a mutant having all the identifying characteristics thereof.

In some embodiments, the presently disclosed subject matter is directed to a method of preparing a feed composition for fish. Particularly, the method comprises adding to the feed composition a formulation comprising a xylanase enzyme and a biologically pure culture of Bacillus licheniformis strain PWD-1 (Accession No. 53757) or a mutant having all the identifying characteristics thereof. In some embodiments of the method, the formulation further comprises a biologically pure culture of Bacillus amyloliquefaciens strain Ba-BPD1 (Accession No. DSM 21836) or a mutant having all the identifying characteristics thereof.

### BRIEF DESCRIPTION OF THE DRAWINGS

FIG. 1a is a photograph of 3 agar diffusion assays showing the interaction between Listeria innocua and B. licheniformis strain PWD-1.

FIG. 1b is a photograph of 3 agar diffusion assays showing the interaction between Salmonella enterica and B. licheniformis strain PWD-1.

FIG. 2a is a photograph of 3 agar diffusion assays showing the interaction between E. coli and B. amyloliquefaciens strain Ba-BPD1.

FIG. 2b is a photograph of 3 agar diffusion assays showing the interaction between Salmonella enterica and B. amyloliquefaciens strain Ba-BPD1.

FIG. 2c is a photograph of 3 agar diffusion assays showing the interaction between Listeria innocua and B. amyloliquefaciens strain Ba-BPD1.

FIG. 3a is a line graph illustrating the growth of B. licheniformis strain PWD-1 over 24 hours after incubation at pH 3.0 for 0, 1, 2, or 3 hours.

FIG. 3b is a line graph illustrating the growth of B. amyloliquefaciens strain Ba-BPD1 over 24 hours after incubation at pH 3.0 for 0, 1, 2, or 3 hours.

### DETAILED DESCRIPTION

The presently disclosed subject matter now will be described more fully hereinafter with reference to the accompanying Figures, in which some (but not all) embodiments are shown. Many modifications and other embodiments of the presently disclosed subject matter set forth herein will come to mind to one skilled in the art having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Unless otherwise defined, all technical terms used herein have the same meaning as commonly understood by one of ordinary skill in the art to which this disclosure belongs.

Following long-standing patent law convention, the terms “a,” “an,” and “the” refer to one or more when used in this application, including the claims. Thus, for example, reference to “a protein” includes a plurality of proteins, unless the context clearly is to the contrary.

For the purposes of this specification and appended claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Throughout this specification and the claims, the terms “comprise,” “comprises,” and “comprising” are used in a non-exclusive sense, except where the context requires otherwise. Likewise, the terms “include” and “have” and grammatical variants are intended to be non-limiting, such that recitation of items in a list is not to the exclusion of other like items that can be substituted or added to the listed items.

The presently disclosed subject matter relates to feed additive formulations for use with fish feed. Particularly, the disclosed formulations comprise an isolated xylanase enzyme and at least one microbial probiotic strain Bacillus licheniformis PWD-1 (Accession No. 53757), or a mutant thereof having all the identifying characteristics thereof. Embodiments of the formulation are described in International Application Publication No. WO 2018/165252, which is incorporated by reference herein in its entirety. The disclosed formulation can be added to fishfeeds, resulting in unexpected synergistically improved fish characteristics as set forth in more detail herein below.

WO 2018/165252 describes using the formulations described therein as additives to animal feed, in particular monogastric aminal feed. Surprisingly, as will be described herein, the same formulations can be used to supplement fish feed to provide improved fish characteristics. The anatomy of fish is quite different from that of mammals. For example, fish lack several organs that are found in mammals, e.g., pancreas, distinct adrenal glands, lymph nodes, lungs, bone marrow and parathyroids. Other organs may be present in fish, but are distinctively different in form and function to their mammalian counterpart, e.g., kidneys, gonads, skin, heart. Additionally, other anatomical features are present in fish but not in mammals or birds. These include fins, the lateral line organ, swim bladder, and the gills. Thus, it is unexpected that formulations that provide improved characteristics in animals would also provide improved characteristics in fish.

The gills and the kidney make up the major excretory organs of fish. The end product of nitrogen metabolism in fish is ammonia. The gills can excrete up to 75% of the ammonia load. Some fish also have a urinary bladder. While some fish have a fully functional stomach, certain other stomachs lack histological differentiation and serve purely as a storage organ. In general herbivorous fish have longer GI tracts than carnivorous fish. Since fish are poikilothermic digestion is very dependent on the environmental (water) temperature.

The gills are one of the main excretory organs. The gills excrete the majority of the ammonia while the rest of the waste products are excreted via the kidneys. The excretion of metabolic waste products is similar in all fish; however, the kidney and the gills play different roles in fresh water fish in comparison to their roles in salt water fish.

The freshwater fish is hypertonic in comparison to the environment. As a direct consequence, water is constantly entering the fish's body via the gills and diluting the blood. Therefore in fresh water fish, the major role of the kidneys is to eliminate the excess water from the circulatory system. In addition, electrolytes must be conserved during the elimination process. Freshwater fish therefore have relatively large glomeruli within the kidney. The situation is exactly the opposite in marine fish. The salt water fish is hypotonic in comparison to the marine environment. Marine fish constantly need to drink water, as water is constantly lost from the gills into the environment. The major work of the kidney is therefore to conserve water and to eliminate electrolytes. For that reason certain marine fish have a glomerulic kidneys.

Thus, the disclosed feed additive formulation comprises an isolated xylanase enzyme as an essential component. The term “xylanase” as used herein refers to a class of enzymes that degrade the linear polysaccharide beta-1,4-xylan into xylose, thus breaking down hemicellulose, one of the major components of plant cell walls. In some embodiments, the xylanase is an endo-1,4-beta-xylanase. The term “isolated” as used herein refers to an enzyme that is substantially pure (i.e., free of contaminating molecules).

Xylanase enzymes suitable for use in the disclosed formulation can be produced using methods well known in the art. For example, in some embodiments, the xylanase can be produced by solid or submerged culture, including batch, fed-batch, and continuous-flow processes. Alternatively or in addition, the xylanase can be any commercially available xylanase. The xylanase can be provided as a liquid or a dry (powder) preparation.

The xylanase can be obtained from any suitable source known or used in the art, such as from a bacterium selected from Bacillus, Streptomyces, Clostridium, Thermonospora, Trichoderma, Thermomyces, Aspergillus, Penicillium, Microtetra-spora, Ruminococcus, and the like. Alternatively or in addition, the xylanase can be obtained from a fungus selected from Trichoderma, Aspergillus, Humicola, Neocallimastix, and the like.

In some embodiments, the xylanase is stable and active at a pH and temperature at or close to the conditions found in the gastrointestinal tract of a fish.

The disclosed formulation further comprises a biologically pure culture of the microbial Bacillus licheniformis strain PWD-1 (Accession No. 53757) or a mutant having all of the identifying characteristics thereof. Bacillus licheniformis strain PWD-1 is described in U.S. Pat. Nos. 4,908,220 and 4,959,311, the entire disclosures of which are hereby incorporated by reference. In some embodiments, the disclosed formulation further comprises a biologically pure culture of Bacillus amyloliquefaciens strain Ba-BPD1 (Accession No. DSM 21836) or a mutant having all of the identifying characteristics thereof. Bacillus amyloliquefaciens strain Ba-BPD1 is described in U.S. Patent Application Publication No. 2010/0143316, the entire content of which is hereby incorporated by reference.

The term “biologically pure culture” refers to a culture that is physically separated from microorganisms of different characteristics. As used herein, the phrase “a biologically pure culture of a bacterial strain” refers to one or a combination of: spores of the biologically pure fermentation culture of a bacterial strain, vegetative cells of the biologically pure fermentation culture of a bacterial strain, one or more products of the biologically pure fermentation culture of a bacterial strain, a culture solid of the biologically pure fermentation culture of a bacterial strain, a culture supernatant of the biologically pure fermentation culture of a bacterial strain, an extract of the biologically pure fermentation culture of the bacterial strain, and one or more metabolites of the biologically pure fermentation culture of a bacterial strain.

The term “mutant” as used herein refers to a genetic variant derived from the parent strain (i.e., Bacillus licheniformis strain PWD-1 or Bacillus amyloliquefaciens strain Ba-BPD1). In some embodiments, the mutant performs as well as or better than the parent strain (e.g., maintains or improves the growth of an animal as well as or better than the parent strain).

The Bacillus licheniformis and/or Bacillus amyloliquefaciens strains can be obtained from research labs or from culture collections. A biologically pure culture can be produced using methods well known in the art, such as by cultivation in a culture-specific medium using aseptic technique and under appropriate conditions (i.e., pH, temperature, oxygen level, and the like).

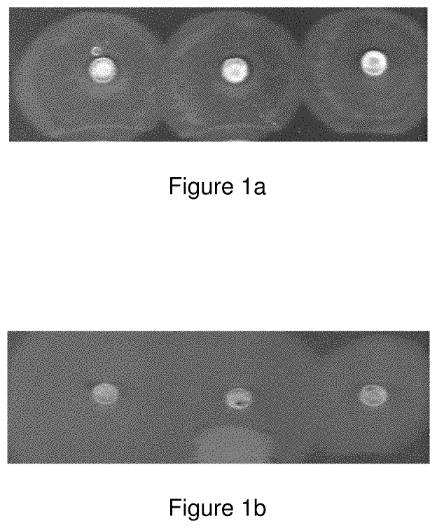

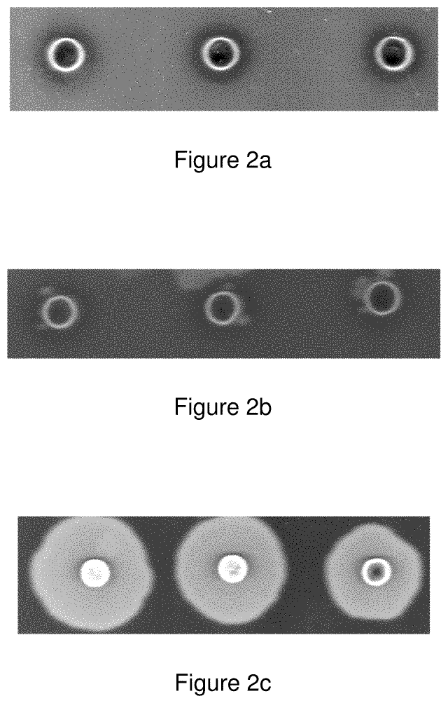

EXAMPLES 1 and 2 of the present disclosure show that the B. licheniformis strain PWD-1 and the B. amyloliquefaciens strain Ba-BPD1 inhibit the growth of human and animal pathogens including Listeria innocua, a E. coli and Salmonella enteric. FIGS. 1a and 1b illustrate that B. licheniformis strain PWD-1 overgrows Listeria innocua and Salmonella enterica, respectively. FIGS. 2a and 2b illustrate that B. amyloliquefaciens strain Ba-BPD1 inhibits the growth of E. coli and Salmonella enterica, respectively. FIG. 2c illustrates that B. amyloliquefaciens strain Ba-BPD1 outgrows Listeria innocua.

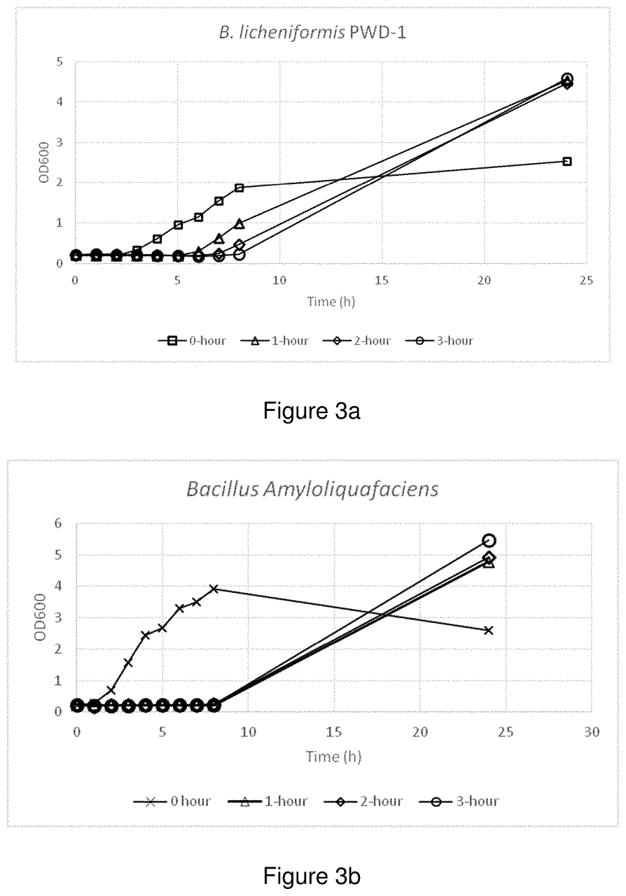

EXAMPLE 3 of the present disclosure shows that the B. licheniformis strain PWD-1 and the B. amyloliquefaciens strain Ba-BPD1 are resistant to acidic environments which is an important feature of probiotic microorganisms due to exposure of the microbial strain to the harsh acidic conditions present in the gut of an animal before passing into the intestine. As shown in FIG. 3a, the exposure of B. licheniformis strain PWD-1 to an acidic environment delayed growth for 3 hours compared to neutral media. It was also observed that B. licheniformis strain PWD-1 exposed to the acidic environment for 1, 2, and 3 hours was able to recover and assume growth. As shown in FIG. 3b, B. amyloliquefaciens strain Ba-BPD1 was also able to recover from the acid exposure, although slower than observed for the B. licheniformis strain.

Feed additive formulations for addition to feed compositions for fish are provided herein. The feed additive formulations are provided to add to feed compositions to improve the health and/or performance of fish such as, for example, Nile tilapia. The increase in performance includes one or a combination of: improved body weight, improved growth performance, specific growth rate, feed conversion ratio, protein efficiency, whole-body nutrient retention, including retention of protein and energy, and nutrient digestibility. The pathogens include, but are not limited to one or a combination of Clostridium perfringens, Eimeria spp., Eimeria acervulina, Eimeria maxima, Eimeria tenella, Salmonella, or Coccidiosis-inducing parasites. In one embodiment, the animal is fish and the increase in performance comprises one or more of improved body weight, improved growth performance, specific growth rate, feed conversion ratio, protein efficiency, whole-body nutrient retention, retention of protein and energy, and nutrient digestibility.

In one embodiment, a feed additive formulation for fish feed is provided comprising an isolated xylanase enzyme and a biologically pure culture of a Bacillus licheniformis strain PWD-1 (Accession No. 53757), or a mutant thereof having all the identifying characteristics thereof. The feed additive formulation may further comprise a biologically pure culture of a Bacillus amyloliquefaciens strain Ba-BPD1 (Accession No. DSM 21836), or a mutant thereof having all the identifying characteristics thereof.

In one embodiment, a feed composition for fish is provided that comprises the disclosed feed additive formulations.

In one embodiment, a method of preparing a feed composition for fish is provided, comprising adding to a feed composition a formulation comprising a xylanase enzyme and a biologically pure culture of a Bacillus licheniformis strain PWD-1 (Accession No. 53757), or a mutant thereof having all the identifying characteristics thereof. In the method, the formulation may further comprise a biologically pure culture of a Bacillus amyloliquefaciens strain Ba-BPD1 (Accession No. DSM 21836), or a mutant thereof having all the identifying characteristics thereof.

In one embodiment, methods are provided for increasing the performance of a fish comprising: administering to the fish an effective amount of a feed composition comprising a xylanase enzyme and a biologically pure culture of a Bacillus licheniformis strain PWD-1 (Accession No. 53757), or a mutant thereof having all the identifying characteristics thereof. The feed composition may further comprise a biologically pure culture of a Bacillus amyloliquefaciens strain Ba-BPD1 (Accession No. DSM 21836), or a mutant thereof having all the identifying characteristics thereof.

In one embodiment, the xylanase is present in the disclosed formulation in an amount ranging from about 10,000-200,000 units/gram. The xylanase can be present in the disclosed formulation in an amount ranging from about 30,000-200,000 units/gram. Thus, the xylanase can be present in an amount of about 10,000; 20,000; 30,000; 40,000; 50,000; 60,000; 70,000; 80,000; 90,000; 100,000; 110,000; 120,000; 130,000; 140,000; 150,000; 160,000; 170,000; 180,000; 190,000; or 200,000 units/gram. One unit of xylanase activity is defined as the amount of enzyme needed for the release of 1 nanomole of reducing sugars (xylose equivalents) per second from 0.5% Xylan (Sima X4252, from Beechwood) at 50° C. in 50 mM trisodium citrate buffer pH 6.0.

In some embodiments, the Bacillus licheniformis strain is present in the disclosed formulation in an amount of from about 10⁸ to 10¹² CFU/gram (colony forming units/gram). In some embodiments, the Bacillus licheniformis strain is present in the disclosed formulation in an amount of at least about 10⁹ CFU/gram. In some embodiments, the Bacillus amyloliquefaciens and Bacillus licheniformis strains are present in the disclosed formulation in an amount of from about 10⁸ to 10¹² CFU/gram. In some embodiments, the Bacillus amyloliquefaciens and Bacillus licheniformis strains are present in the disclosed formulation in an amount of at least about 10⁹ CFU/gram. Thus, the strains can be present in the disclosed formulation in an amount of at least about 10⁸, 10⁹, 10¹⁰, 10¹¹, 10¹², or 10¹³ CFU/gram.

In addition to the xylanase and the bacterial strain(s), the disclosed formulation can further comprise a carrier to improve production, stability, and/or performance characteristics. The term “carrier” as used herein refers to an edible material to which ingredients are added to facilitate uniform incorporation of the ingredients into the disclosed formulation. Suitable carriers can include (but are not limited to) limestone, maltrodextrin, cyclodextrin, wheat, and combinations thereof. In some embodiments, the ratio of active ingredient to carrier can be about 1:9, 1:8, 1:7, 1:6, 1:5, 1:4, 1:3, 1:2, 1:1, 2:1, 3:1, 4:1, 5:1, 6:1, 7:1, 8:1, or 9:1.

The disclosed formulation can be in any desired form, including (but not limited to) a solid, powder, suspension concentration, liquid, or granule.

In some embodiments, the disclosed formulation can be thermally stable to heat treatment up to about 70° C., 80° C., 85° C., 90° C., or 95° C. for a period of up to about 1, 5, 10, 15, 30, or 60 minutes. As used herein, the term “thermally stable” indicates that at least 75% of the components present in the formulation before heating to the specified temperature are still present after it cools to room temperature.

In some embodiments, the disclosed formulation can have a shelf life of greater than 30, 40, 50, 60, 70, or 80 weeks. It should be understood that the desired length of time and normal shelf life can vary upon the storage temperatures, processing conditions, packaging material, packaging equipment, and the like.

The disclosed formulation can be added to a feed composition for consumption by fish. The formulation can be mixed directly with the fish feed and/or can be mixed with a feed additive (i.e. vitamin feed additive, mineral feed additive, amino acid feed additive, and the like) that is then mixed with the fish feed.

“Fish feed” or “feed” as used herein refers to any compound, preparation, mixture, or composition suitable for or intended for intake by a fish. In some embodiments, the feed can comprise a fish feed composition. The term “fish” as used herein includes any of various cold-blooded, aquatic vertebrates, having gills, and typically an elongated body covered with scales. In embodiments, fish may include those suitable for use in aquaculture, such as fish farming. In some embodiments, suitable fish can include (but are not limited to) fish species suited to closed recirculating systems such as: tilapia, including Nile tilapia and Blue tilapia; striped bass (Morone saxatilis), also referred to as Atlantic striped bass, linesider, rock or rockfish; barramundi or Australian sea bass (Lates calcarifer); yellow perch (Perca flavescens), also referred to as perch, striped perch, American perch or preacher; sturgeon (family Acipenseridae) and eel (order Anguilliformes). Other suitable fish species, which are commonly raised in outdoor pond systems, can include tilapia (e.g., Nile tilapia—Oriochromis niloticus); yellow perch; walleyes (Sander vitreus), also referred to as yellow pike or yellow pickerel; trout (such as brook (Salvelinus fontinalis), brown, and rainbow trout); catfish (Siluriformes); largemouth bass (Micropterus salmoides); koi/carp; shiners, including common shiner (Luxilus cornutus) and golden shiner (Notemigonus crysoleucas); bluegills (Lepomis macrochirus), also referred to as bream, brim, sunny or copper nose; and sunfish (family Centrarchidae). Suitable fish species also include salmon (Salmo salar).

In some embodiments, the disclosed formulation can be added to fish feed. Thus, the disclosed formulation can be added to fish feed to produce a feed containing a desired amount of xylanase and the disclosed bacterial strain(s). As would be understood to those of ordinary skill in the art, the dilution amount can be determined by the feed needs of the fish, age of the fish, and intended use. For example, in some embodiments, the concentration of xylanase ranges from about 5 to 30 units/gram feed. In some embodiments, the concentration of xylanase ranges from about 7.5 to 30 units/gram feed (i.e., about 5.0, 5.5, 6.0, 6.5, 7.0, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 units/gram feed). In some embodiments, the amount of Bacillus licheniformis in the feed composition ranges from at least about 5×10⁴ CFU/gram feed. In some embodiments, the amount of Bacillus licheniformis in the feed composition ranges from at least about 10⁵ CFU/gram. In some embodiments, the amount of Bacillus licheniformis in the feed composition ranges from at least about 10⁶ CFU/gram. In some embodiments, the feed composition further comprises the disclosed Bacillus amyloliquefaciens strain at a concentration of at least about 10⁵ CFU/gram feed. In some embodiments, the feed composition comprises a total concentration of both strains of at least about 10⁵ CFU/gram feed. In some embodiments, the feed composition comprises a total concentration of both strains of at least about at least about 2.0×10⁵ CFU/gram.

When administered to a fish, an effective amount of a feed composition comprising the disclosed formulation has been shown to increase the performance of the animal compared to feed compositions that lack the disclosed formulation. The term “effective amount” refers to the amount of feed sufficient to increase performance in fish without resulting in any significant adverse side effects.

In some embodiments, the increased performance comprises one or more of improved body weight, improved growth performance, specific growth rate, feed conversion ratio, protein efficiency, whole-body nutrient retention, retention of protein and energy, and nutrient digestibility. The improved body weight refers to the total weight gain of all fish over a time period.

### EXAMPLES

The following EXAMPLEs have been included to provide guidance to one of ordinary skill in the art for practicing representative embodiments of the presently disclosed subject matter. In light of the present invention and the general level of skill in the art, those of skill can appreciate that the following EXAMPLEs are intended to be exemplary only and that numerous changes, modifications, and alterations can be employed without departing from the scope of the presently disclosed subject matter.

### Example 1

### Assessment of B. licheniformis Strain PWD-1 Probiotic Characteristic In Vitro

B. licheniformis strain PWD-1 was tested against the pathogen surrogates E. coli, Salmonella enterica, Listeria innocua, and Campylobacter hyointestinalis via a series of agar well diffusion assays. In each assay, one of the pathogen surrogates was spread onto 3 agar plates and B. licheniformis strain PWD-1 was put into the well of each plate. The plates were then incubated overnight at 37° C. and photographed to show the interaction between the pathogens and the B. licheniformis.

FIGS. 1a and 1b illustrate that B. licheniformis strain PWD-1 overgrows Listeria innocua and Salmonella enterica, respectively.

### Example 2

### Assessment of B. amyloliquefaciens Strain Ba-BPD1 Probiotic Characteristic In Vitro

The experiment of Example 1 was repeated using B. amyloliquefaciens strain Ba-BPD1 in place of B. licheniformis strain PWD-1.

FIGS. 2a and 2b illustrate that B. amyloliquefaciens strain Ba-BPD1 inhibits the growth of E. coli and Salmonella enterica, respectively. FIG. 2c illustrates that B. amyloliquefaciens strain Ba-BPD1 outgrows Listeria innocua.

### Example 3

### Acid Resistance Study of B. licheniformis Strain PWD-1 and B. amyloliquefaciens Strain Ba-BPD1

The resistance of probiotic microorganisms to acidic environments is an important feature due to exposure of the probiotic to the harsh acidic conditions present in the gut of an animal before passing into the intestine. B. licheniformis strain PWD-1 was subjected to LB media at pH 3.0 for time points of 0, 1, 2, and 3 hours. The media was then neutralized to pH 7.0 and growth was monitored by absorbance at 600 nm. The experiment was repeated for B. amyloliquefaciens strain Ba-BPD1.

As shown in FIG. 3a, the exposure of B. licheniformis strain PWD-1 to an acidic environment delayed growth for 3 hours compared to neutral media. It was also observed that B. licheniformis strain PWD-1 exposed to the acidic environment for 1, 2, and 3 hours was able to recover and assume growth. As shown in FIG. 3b, B. amyloliquefaciens strain Ba-BPD1 was also able to recover from the acid exposure, although slower than observed for the B. licheniformis strain.

### Example 4

### Investigation of the Effects of the Addition of Xylanase and B. licheniformis Strain PWD-1 to the Diet of Juvenile Nile Tilapia (Oreochromis niloticus)

The effect of a blend of endo-xylanase and multi-strain Bacillus spp. DFMs (EP), on growth performance, whole body nutrient retention and nutrient digestibility in juvenile Nile tilapia (Oreochromis niloticus) was evaluated.

600 juvenile tilapia, with mean initial body weight (BW) of 12.3±0.7 g, were randomly assigned to 1 of 3 dietary treatments, with 4 replicate tanks of 50 fish per treatment, and raised to 61 days of age in 500 L recirculating freshwater tanks. The first dietary treatment was a standard-energy practical diet (positive control, PC) formulated to 4326 kcal/kg gross energy (GE), 32.8% crude protein and 8.6% crude fat. In the second treatment (NC), dietary energy was reduced by 120 kcal/kg GE (2.1% reduction in crude fat), compared to the PC. In the third treatment, NC diets were supplemented with 100 g/MT xylanase-DFM blend (NC+EP). All diets contained fishmeal (5% of the diet) and plant-based ingredients as protein and fiber sources. Fish were hand-fed to satiety 3 times/day. Table 1 shows the details of the three dietary treatments.

TABLE 1
Calculated analysis of experimental diets.

| | PC | NC | NC + EP |
|---|---|---|---|
| Crude protein, % | 32.8 | 32.8 | 32.8 |
| Crude lipid, % | 8.6 | 6.5 | 6.5 |
| Gross energy (kcal/kg) | 4326 | 4206 | 4206 |
| Total phosphorus, % | 1.11 | 1.08 | 1.10 |
| Xylanase activity (U/g) | — | — | 10.0 |
| Bacillus spp. (CFU/g) | — | — | 1.0 × 10⁵ |

After 61 days, the third dietary treatment, which supplemented NC diets with EP, significantly improved (P<0.05) final body weight, specific growth rate, feed conversion ratio, protein efficiency, whole-body retention of protein and energy, and apparent digestibility of energy in the tested fish. At 100 g/MT, EP supplemented to NC diets improved growth performance, feed efficiency and digestibility of key nutrients at a rate similar to, or exceeding, the first dietary treatment (standard-energy PC diets). Thus, in testing, the third dietary treatment (EP supplemented to NC diets) compensated for at least 120 kcal/kg GE in juvenile tilapia diets. The results of the testing are shown in Table 2.

TABLE 2
Tilapia growth performance, whole-body nutrient retention and apparent
digestibility of energy after 61 days feeding. Means within rows
with different letters differ significantly (P < 0.05).

| Metric | PC | NC | NC + EP |
|---|---|---|---|
| Final body weight, g | 70.1 b | 63.9 a | 72.0 b |
| Specific growth rate, %/d | 2.86 b | 2.70 a | 2.89 b |
| Feed conversion ratio | 1.07 a | 1.18 b | 1.08 a |
| Protein efficiency ratio | 2.85 b | 2.59 a | 2.82 b |
| Protein retention, % | 39.5 b | 35.8 a | 38.9 b |
| Energy retention, % | 30.5 b | 28.3 a | 30.7 b |
| App. dig. of energy, % | 87.8 a | 87.1 a | 89.3 b |

1-24. (canceled)

25. A fish feed additive formulation, the formulation comprising an isolated xylanase enzyme and a biologically pure culture of a Bacillus licheniformis strain PWD-1 (Accession No. 53757), or a mutant thereof having all the identifying characteristics thereof.

26. The fish feed additive formulation of claim 25, further comprising a biologically pure culture of a Bacillus amyloliquefaciens strain Ba-BPD1 (Accession No. DSM 21836), or a mutant thereof having all the identifying characteristics thereof.

27. The fish feed additive formulation of claim 25, wherein the xylanase is an endo-1,4-beta-xylanase.

28. The fish feed additive formulation of claim 25, wherein the formulation is a solid, a powder, a suspension concentrate, a liquid, or a granule.

29. The fish feed additive formulation of claim 25, further comprising a carrier.

30. The fish feed additive formulation of claim 29, wherein the carrier comprises limestone.

31. The fish feed additive formulation of claim 25, wherein the xylanase ranges from 10,000-200,000 units/gram and the Bacillus licheniformis ranges from 10⁸-10¹² CFU/gram.

32. The fish feed additive formulation of claim 26, wherein the xylanase ranges from 10,000-200,000 units/gram, the Bacillus licheniformis ranges from 10⁸-10¹² CFU/gram, and the Bacillus amyloliquefaciens ranges from 10⁸-10¹² CFU/gram.

33. A fish feed composition comprising the feed additive formulation of claim 25.

34. The fish feed composition of claim 33, further comprising a biologically pure culture of a Bacillus amyloliquefaciens strain Ba-BPD1 (Accession No. DSM 21836), or a mutant thereof having all the identifying characteristics thereof.

35. The fish feed composition of claim 33, wherein the xylanase ranges from 5-30 units/gram feed and the Bacillus licheniformis ranges from at least 5×10⁴ CFU/gram feed.

36. The fish feed composition of claim 34, wherein the xylanase ranges from 5-30 units/gram feed and each of the Bacillus licheniformis and the Bacillus amyloliquefaciens ranges from at least 10⁵ CFU/gram feed for a total of at least 1×10⁵ CFU/gram feed.

37. A method for increasing the performance of fish comprising: administering to the fish an effective amount of a feed composition comprising a xylanase enzyme and a biologically pure culture of a Bacillus licheniformis strain PWD-1 (Accession No. 53757), or a mutant thereof having all the identifying characteristics thereof, wherein the increase in performance comprises one or a combination of: improved body weight, improved growth performance, specific growth rate, feed conversion ratio, protein efficiency, whole-body nutrient retention, retention of protein and energy, and nutrient digestibility.

38. The method of claim 37, wherein the feed composition further comprises a biologically pure culture of a Bacillus amyloliquefaciens strain Ba-BPD1 (Accession No. DSM 21836), or a mutant thereof having all the identifying characteristics thereof.

39. The method of claim 37, wherein the xylanase is an endo-1,4-beta-xylanase.

40. The method of claim 37, wherein the fish is selected from one or more of tilapia, including Nile tilapia (Oriochromis niloticus) and Blue tilapia; striped bass (Morone saxatilis), also referred to as Atlantic striped bass, linesider, rock or rockfish; barramundi or Australian sea bass (Lates calcarifer); yellow perch (Perca flavescens), also referred to as perch, striped perch, American perch or preacher; sturgeon (family Acipenseridae) and eel (order Anguilliformes); yellow perch; walleyes (Sander vitreus), also referred to as yellow pike or yellow pickerel; trout (such as brook (Salvelinus fontinalis), brown, and rainbow trout); catfish (Siluriformes); largemouth bass (Micropterus salmoides); koi/carp; shiners, including common shiner (Luxilus cornutus) and golden shiner (Notemigonus crysoleucas); bluegills (Lepomis macrochirus), also referred to as bream, brim, sunny or copper nose; sunfish (family Centrarchidae); and salmon (Salmo salar).

41. A method of preparing a feed composition for fish, comprising adding to the feed composition a formulation comprising a xylanase enzyme and a biologically pure culture of a Bacillus licheniformis strain PWD-1 (Accession No. 53757), or a mutant thereof having all the identifying characteristics thereof.

42. The method of claim 41, wherein the formulation further comprises a biologically pure culture of a Bacillus amyloliquefaciens strain Ba-BPD1 (Accession No. DSM 21836), or a mutant thereof having all the identifying characteristics thereof.

43. The method of claim 41, wherein the xylanase ranges from 5-30 units/gram feed and the Bacillus licheniformis ranges from at least 5×10⁴ CFU/gram feed.

44. The method of claim 41, wherein the xylanase ranges from 5-30 units/gram feed and each of the Bacillus licheniformis and the Bacillus amyloliquefaciens ranges from at least 10⁵ CFU/gram feed for a total of at least 1×10⁵ CFU/gram feed.
